(19) United States
(12) Patent Application Publication
(10) Pub. No.: US 2004/0267221 A1
(43) Pub. Date: Dec. 30, 2004

(54) Newborn nappy

(76) Inventor: Robin Maria Fletcher, Hamilton (NZ)

Correspondence Address: BLAKELY SOKOLOFF TAYLOR & ZAFMAN, 12400 WILSHIRE BOULEVARD, SEVENTH FLOOR, LOS ANGELES, CA, 90025-1030, US

(21) Appl. No.: 10/485,122

(22) PCT Filed: Jul. 25, 2002
(86) PCT No.: PCT/NZ02/00131

(30) Foreign Application Priority Data
Jul. 25, 2001 (NZ) 513119

(51) Int. Cl. A61F 13/15; A61F 13/20

(52) U.S. Cl. 604/385.01

(57) ABSTRACT

An infant nappy (1) configured to provide releasable envelopment of an infant's pelvis, said nappy (1) having a centre crotch section (4) interposed between a front portion (2) and a rear portion (3), said front (2) and rear portions (3) being mutually and releasably fastenable, characterised in that, said front portion (2) includes a recess (10) located on an inner nappy surface (14) opposing a navel region (11) of an infant when wearing said nappy (1), said recess (10) being capable of enclosing said navel region (11) and accommodating a newborn infant's post-delivery residual umbilical cord (12).

### CROSS-REFERENCE TO OTHER APPLICATIONS

This Application is a National Phase of International Application No.

PCT/NZ02/00131, filed on Jul. 25, 2003, which claims priority from New Zealand Patent Application No. 513119, filed on Jul. 25, 2001, and New Zealand Patent Application No. 513119, filed on Jan. 9, 2002.

### TECHNICAL FIELD

The present invention relates generally to the field of sanitary nappies, diapers and the like, and in particular to those for newborn infants.

### BACKGROUND ART

The advent of the disposable nappy, or diaper has brought significant advantages in both convenience and hygiene over the re-usable products previously employed.

Due to proportionally rapid growth rate of human infants, most disposable nappy manufacturers produce a range of product sizes (often gender specific) for an optimised fit as the child grows. The product range is typically composed of a plurality of nappy designs intended to cover a particular age or infant weight range.

There is typically a degree of overlap in the sizing capability of adjacent products within such a range. However, nappies for newborn infants obviously do not have any overlapping capacity with nappies intended for smaller infants. Furthermore, the period immediately following the infant birth corresponds to the largest proportional size increase in the infant and thus nappies for newborn infants are typically only usable for a narrow weight/size range.

This can lead caregivers into using oversize (albeit cheaper and more readily available) nappies for newborn infants as an economic and practical compromise to the use of specialised newborn nappies.

Naturally, this leads to a reduction in performance of the nappy, potentially causing chaffing of the infant through poor fit, and/or leakage of faecal and urinal body products. These difficulties may be further exacerbated in the case of premature or significantly underweight infants.

A further factor common to all newborn infants affecting the usage of nappies is the umbilicus, whereby the newly severed residue umbilical cord provides a potential infection and/or inflammation sight. Use of inappropriately designed or sized nappies can lead to numerous difficulties such as:

frictional irritation of the umbilical cord by direct contact with the nappy liner or, if the nappy does not cover the umbilical cord, by contact with the garments clothing the infant; and/or

transmission of faeces and urine to the umbilical region via a body movement of the infant in the absence of correctly sized/designed physical barriers within the nappy lining.

Current thinking regarding the treatment of the umbilical cord site with respect to sanitary nappies falls into two main schools of thought. On the one hand, the umbilical cord is left uncovered by the nappy. Alternatively, the umbilical cord and surrounding portion of the infant's torso is covered by a relatively large nappy.

Leaving the umbilical cord uncovered does accelerate the time required for the cord to dry out and detach from the umbilicus. Various prior art solutions following this philosophy such as U.S. Pat. No. 4,230,113, (Mehta) have utilised v-shaped, or u-shaped notches in the upper front portion of the nappy to leave the umbilical cord exposed. U.S. Pat. No. 4,675,015 (Brown) incorporates a separable opening through which the umbilical cord may project. U.S. Pat. No. 4,769,023 Goebel et al provides a diaper with “removable perforated area” of various configurations which may be removed to leave the umbilicus uncovered by the diaper.

U.S. Pat. No. 4,801,298 Sorenson et al discloses a re-useable diaper in which the upper front panels are attached together with “V”-shaped connecting strips. In one embodiment, the connecting strips may be joined together below the infants navel to avoid placement of fabric on the umbilicus during the sensitive period.

However, such nappy configurations do not obviate the lack of protection of the clamped umbilical cord from being snagged on the infant's clothing, or from unintentional impact by the infant, or caregiver during handling of the child.

French Patent 2604867 A1 Gamas discloses a nappy with a cut-out portion at the upper edge of the front of the nappy in which a “compress” is located.

The cut-out region is edged by a protective band separating and sealing the nappy from the compress by virtue of the band's extra thickness with respect to the main body of the nappy. The disclosure cites the advantage of dispensing with the need to change both the nappy and an umbilical compress during the first 10 days of an infants life. Effectively, the compress is built into the nappy.

However, current thinking regarding care of umbilicus dispenses with the need for a compress. Furthermore, the specification does not disclose any means of affording physical protection to the umbilicus and/or navel region in general. The outer fabric and absorbent pad/cushioning material of the nappy itself does not extend over the top of the compress/navel region. Thus the umbilicus would be vulnerable to irritation during handling by caregivers, movement of the child and/or inadvertent contact with external objects.

The alternative approach of covering the umbilicus with a conventionally designed nappy is also fraught with undesirable drawbacks. In order to adequately cover the entire umbilical area (including the un-detached umbilical cord and clamp) and provide a sufficient margin with the nappy perimeter to prevent any protrusion of the cord a nappy dimensioned with a disproportionally high front section is required. In practice, nappies sold as suitable for newborn infants are simply scaled-down versions of larger conventional proportioned nappies.

Consequently, the un-detached umbilical cord is either enclosed by the nappy beneath the nappy liner surface, or the upper front section of the nappy is folded (inwards or outwards) to expose the umbilical cord as per the former approach.

Neither approach adequately protects the umbilicus from infection, irritation, abrasion and trauma. Clearly, any discomfort experienced by the infant as a consequence of these defects will cause unwanted anxiety in both the infant and caregivers.

It is an object of the present invention to address the foregoing problems or at least to provide the public with a useful choice.

Further aspects and advantages of the present invention will become apparent from the ensuing description which is given by way of example only.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art, in New Zealand or in any other country.

### DISCLOSURE OF INVENTION

According to one aspect of the present invention there is provided an infant nappy configured to provide releasable envelopment of an infant's pelvis, said nappy having a centre crotch section interposed between a front portion and a rear portion, said front and rear portions being mutually and releasably fastenable, characterised in that,

said front portion includes a recess located spaced-apart from a peripheral edge of said front portion on an inner nappy surface opposing a navel region of an infant when wearing said nappy,

said recess being capable of enclosing said navel region and accommodating a newborn infant's post-delivery residual umbilical cord and, if present, an umbilical cord clamp attached thereto

said front portion having a continuous protective outer surface, extending fully over said recess.

As used herein, the term ‘infant’s pelvis' is defined as including those portions of an infants anatomy conventionally deemed to require coverage in order to provide satisfactory functioning of a nappy as an incontinence or sanitary aid.

Consequently, variations in the precise portions of the infant's torso covered by the nappy insofar as they do not affect the nappy's coverage of the navel region are not germane to the present invention.

Similarly, the term ‘releasably fastenable’ as used herein is defined as including any convenient known fastening means including, but not limited to, hook and loop fasteners, adhesive tabs, pins, and the like, the precise choice of which is not relevant to the inventive aspects of the present invention.

The term ‘navel region’ as used herein, refers to the region centred on a human umbilicus in the central midriff and its immediate surrounds.

Preferably, said recess is formed from breathable materials with a smooth, non-irritating surface. The breathabililty of the nappy in the recess permits the umbilical cord to safely atrophy and eventually fall off, without risk of adverse interference from the surroundings. Presenting a smooth inner nappy surface to the umbilical cord obviously minimises the likelihood of abrasion and inflammation of same.

By locating the recess away from the edges of the front section (particularly the upper edge), further protection is provided for the navel region.

In addition to simply forming a recess on the inner surface of the nappy above the umbilicus, i.e. the said recess, a degree of additional mechanical strengthening may be incorporated. This helps prevent external pressure on the outer surface of the nappy in the vicinity of the umbilicus from bearing on the sensitive umbilical cord and surrounds.

Therefore, according to a further aspect of the present invention there is provided an infant nappy as described above, characterised in that said recess is strengthened with respect to the adjacent front portion.

In order to maintain the hygiene of the navel area and the umbilical cord (if still present), it is desirable to prevent any contact with the infant's urine or faeces.

Consequently, any appropriate manner of physically separating said recess from said crotch section by one or more physical barriers formed on said inner nappy surface may be employed. This may take the form of leak-guards, absorbent barriers, and/or a close physical fit between the inner nappy surface and the infant's skin between the crotch and the said recess.

According to another aspect of the present invention there is provided a method of facilitating hygienic care of, and eventual detachment from a newborn infant of, a terminal portion of a severed umbilical cord, said method

characterised by the steps of, the fitment and removal of a nappy as hereinbefore described according to the sanitary requirements of the infant.

Regular changing of the infant's nappy of the present invention in accordance with conventional sanitary care of an infant, will aid in the hygienic treatment and early removal of the umbilical cord, whilst mitigating biological infection of the umbilicus.

It will be appreciated that the present invention may still be worn by infants as a conventional nappy after the umbilical cord has fallen off.

### BRIEF DESCRIPTION OF DRAWINGS

Further aspects of the present invention will become apparent from the ensuing description which is given by way of example only and with reference to the accompanying drawings in which:

FIG. 1. shows a plan view of the inner surface of a preferred embodiment of the present invention; and

FIG. 2. shows a partial section of a side elevation of the embodiment shown in FIG. 1, being worn by an infant.

### BEST MODES FOR CARRYING OUT THE INVENTION

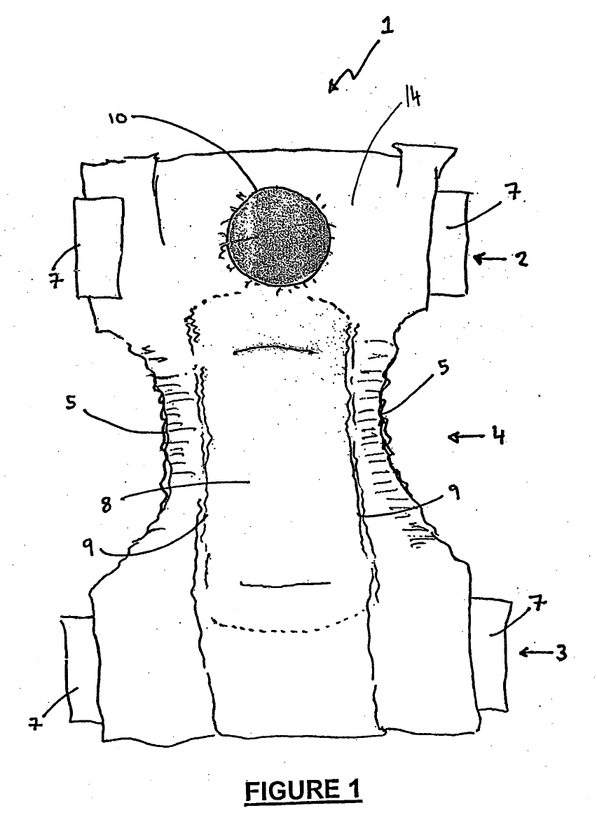

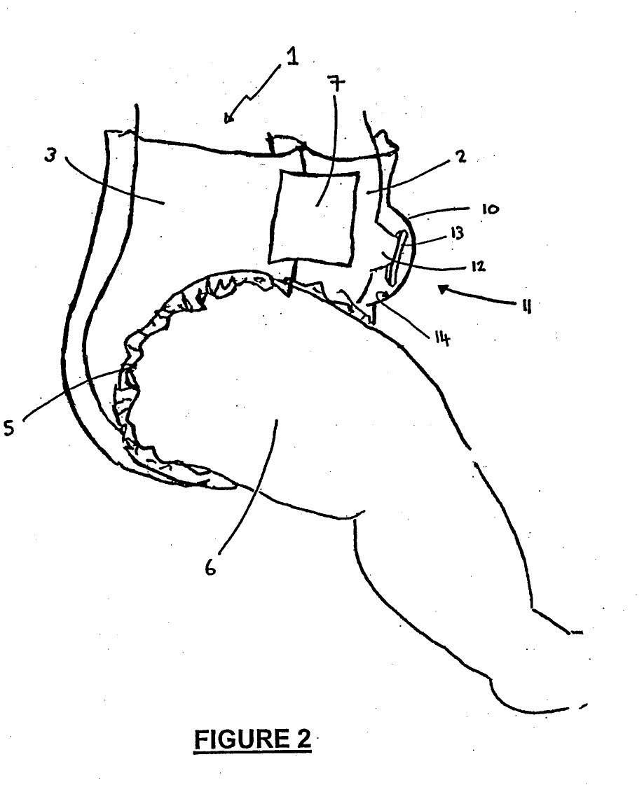

FIGS. 1 and 2, illustrate a preferred embodiment of the present invention in the form of an infant nappy (1) configured to provide umbilicus relief from mechanical irritation and contact with urine or faeces collected by the nappy (1) and to thereby prevent umbilical infection on a newborn baby prior to the healing of the umbilical area on the infant.

The nappy (1) is substantially comprised of a front portion (2), a rear portion (3) and a central crotch portion (4) interposed therebetween. The nappy (1) is substantially rectangular in plan view with arcuate portions (5) adjacent the central crotch portions (4) shaped to circumscribe the legs of an infant (6) as shown in FIG. 2. The front and rear portions (2, 3) are releasably attachable to each other via adhesive tabs (7) projecting from the lateral edges of the front and rear portions (2, 3).

The inner surface (14) of the nappy (1) facing the infant's (6) skin in use is shown in FIG. 1. The inner surface of the central crotch section (4) includes an absorbent section (8) designed to absorb the infant's excreted body fluids. The perimeter of the absorbent section (8) is located within a predetermined distance from the arcuate side portions (5) and protected therefrom by leak-guards (9) in known manner.

The front section (2) also includes a recess (10) on the inner nappy surface (14) located at a point which would overlay the umbilicus, or navel region (11) when the nappy (1) is worn by the infant (6). In the embodiment shown in the drawings, the recess (10) is a circular concave dome shape, though it will be readily appreciated that the invention is not restricted to same. Any convenient shape (e.g. oval, rectangular and so forth) of recess (10) may be employed provided it forms a protective shield over the umbilical cord (12) of a newborn infant (6).

The recess (10) is preferably sufficiently dimensioned to accommodate a clamp (13) attached to the umbilical cord (12). Typically, the umbilical cord (12) remains attached to the infant (6) for a few day following birth, during which time it atrophies and eventually detaches, leaving the formed umbilicus or navel (11).

The recess (10) is physically separated from the absorbent section (8) by a portion of the front portion (2) which lies flush with the infants (6) skin. This provides a physical barrier to the transmission of excrement or urine from the infant (6) to the navel area (11). Furthermore, in the embodiment shown, the recess (10) is spaced apart from the edges of the front portion (2) providing additional protection. The physical protection of the umbilicus (11) from potentially infectious material may be further enhanced by additional leak guards of some form (not shown) extending substantially transversely across the top of the absorbent portion (8).

Further protection of the umbilical cord (12) may be provided by forming the recess (10) with a degree of mechanical strengthening to prevent, or at least mitigate the affects of physical impacts to the outer surface of the nappy (1) in the navel region (11). This strengthening may be accomplished by any appropriate means in known manner, such as the use of stiffened fabric in and around the recess (10). Naturally, the degree of said strengthening must be balanced against creating an overly rigid area which may impede the necessary flexibility of the nappy (1) as a whole.

Aspects of the present invention have been described by way of example only and it should be appreciated that modifications and additions may be made thereto without departing from the scope thereof as defined in the appended claims.

1. An infant nappy configured to provide releasable envelopment of an infant's pelvis, said nappy having a centre crotch section interposed between a front portion and a rear portion, said front and rear portions being mutually and releasably fastenable,
characterised in that,
said front portion includes a recess located spaced-apart from a peripheral edge of said front portion on an inner nappy surface opposing a navel region of an infant when wearing said nappy,
said recess being capable of enclosing said navel region and accommodating a newborn infant's post-delivery residual umbilical cord,
said front portion having a continuous protective outer surface, extending fully over said recess.

2. An infant nappy as claimed in claim 1, characterised in that said recess is also capable of accommodating an umbilical cord clamp attached to said umbilical cord.

3. An infant nappy as claimed in claim 1, characterised in that said recess is formed from breathable materials.

4. An infant nappy as claimed in claim 1, characterised in that said recess provides a smooth, non-irritating surface.

5. An infant nappy as claimed claim 1, characterised in that said recess is strengthened with respect to the adjacent front portion.

6. An infant nappy as claimed in claim 1, characterised in that said recess is separated from said crotch section by one or more physical barriers formed on said inner nappy surface.

7. A method of facilitating hygienic care of, and eventual detachment from a newborn infant of a terminal portion of a severed umbilical cord, said method characterised by the steps of, the fitment and removal of a nappy as claimed in claim 1 according to the sanitary requirements of the infant.

8-9. (Cancelled)
